(19) United States
(12) Patent Application Publication
(10) Pub. No.: US 2025/0331817 A1
(43) Pub. Date: Oct. 30, 2025

(54) PORTABLE IMAGING SYSTEMS, METHODS, AND DEVICES

(71) Applicant: THE BOARD OF REGENTS OF THE UNIVERSITY OF TEXAS SYSTEM, Austin, TX (US)

(72) Inventor: Caroline Park, Dallas, TX (US)

(21) Appl. No.: 19/189,246

(22) Filed: Apr. 24, 2025

Related U.S. Application Data
(60) Provisional application No. 63/637,966, filed on Apr. 24, 2024.

(51) Int. Cl. A61B 8/00; G09B 23/28

(52) U.S. Cl. CPC A61B 8/4405; A61B 8/4218; A61B 8/462; A61B 8/5207; G09B 23/286

(57) ABSTRACT

Implementations claimed and described herein provide systems and methods for performing medical imaging using a portable imaging system. The systems and methods use the portable imaging system to generate an image to assist in providing medical treatment.

### CROSS-REFERENCE TO RELATED APPLICATIONS

The present application claims priority to U.S. Provisional Application No. 63/637,966, entitled “Vascular Access Sheath Extension Systems, Methods, and Devices” and filed on Apr. 24, 2024, which is specifically incorporated by reference in its entirety herein.

### FIELD

Aspects of the present disclosure relate to medical imaging at a location remote from medical treatment facility and more particularly to portable ultrasonic imaging.

### BACKGROUND

Time is a critical factor in responding to an emergency medical event (e.g., a critical injury involving bleeding). As a result, many important actions in providing treatment occur at locations remote from a medical treatment facility, such as a hospital. For example, an emergency medical technician (EMT) team may arrive at a scene of the emergency medical event and take actions necessary to treat the patient at the scene and during transport to a medical treatment facility. However, in cases of critical injury, patients often lose significant amounts of blood while being transported to a medical treatment facility. Locating a target artery, such as a femoral artery, is critical to stop bleeding as fast as possible, but locating the target artery is challenging with the limited resources available at the scene and during transport in an emergency vehicle.

It is with these observations in mind, among others, that various aspects of the present disclosure were conceived and developed.

### SUMMARY

Implementations described and claimed herein address the forgoing by providing systems, methods, and devices for portable medical imaging. In one example implementation, a portable imaging system including a portable imaging device including: a base including a base surface, a mounting post coupled to base and extending from the base surface, an extension arm coupled to the mounting post and including an extension arm surface that cooperatively forms a treatment area with the base surface, and an image generation device mount coupled to at least one of the base, the mounting post, or the extension arm.

In another example implementation, a portable imaging device comprising a base, a mounting post coupled to and extending from the base, an extension arm coupled to the mounting post cooperatively forming a treatment area with the base, and an image generation device mount coupled to at least one of the base, the mounting post, or the extension arm, the image generation device mount configured to couple to an image generation device, the image generation device configured to generate image data.

In another example implementation, a method for generating an image using the portable imaging system, the method comprising generating image data using an image generation device, the image generation device coupled to a portable imaging device via an image generation device mount, the image generation device mount coupled to at least one of a base, a mounting post, or an extension arm of the portable imaging device; processing the image data to generate processed image data; generating output data using the processed image data; and transmitting the output data to a display to cause an output of a representation of the output data.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIGS. 1A-1B illustrate an example system for portable medical imaging.

FIG. 2 illustrates an example portable imaging system.

FIG. 3A illustrates an example training system.

FIG. 3B illustrates an example pump system of the training system of FIG. 3A.

FIG. 4 illustrates example operations for generating portable medical imaging of a patient.

FIG. 5 illustrates an example computing system that may implement various aspects of the systems and methods described herein.

### DETAILED DESCRIPTION

Aspects of the present disclosure generally involve portable medical imaging in response to an emergency medical event, which may include, without limitation, a critical injury, a cardiac event, a stroke, and/or the like. In some aspects, a portable imaging device may be used to evaluate a patient for diagnosis and treatment. The portable imaging device may be configured for deployment at various locations for imaging the patient in-situ during the course of an emergency medical response. For example, the portable imaging device may be used at locations remote from and/or within a medical treatment facility, including at a scene of the emergency medical event (e.g., by an EMT team), during transport from the scene to the medical treatment facility in an emergency vehicle, at locations proximate to or within holding locations at the medical treatment facility during triage, and/or so forth. Stated differently, the portable imaging device facilitates imaging of the patient where the patient is located and as needed, rather than having to wait until large, cumbersome equipment is available and bringing the patient to the equipment. Accordingly, the systems, methods, and devices disclosed herein facilitate timely interventions for a patient through in-situ imaging, including at locations remote from a medical treatment facility that would normally not have access to imaging equipment, thereby increasing patient survival rates and/or treatment success rates.

In some examples, the portable imaging device is configured to provide two-dimensional imaging, such as ultrasonic imaging. Stated differently, the portable imaging device may be configured to emit and detect ultrasonic waves, which may be visualized as one or more images on a display. Ultrasonic imaging may be used to visualize blood flow, including blockages, sources of blood loss, and/or the like, as well as generally visualizing internal anatomical structures of a patient, such as vascular structures, cardiac structures, organs, etc. In doing so, the portable imaging device may be used to diagnose and begin treatment in response to an emergency medical event before a patient arrives at a medical treatment facility. For example, the portable imaging device may capture ultrasound images of a target artery. The target artery may be a source of bleeding. Often, it is challenging to determine where a particular artery is located within patient tissue (e.g., where the femoral artery is located within a patient leg), let alone an artery that is a source of blood loss. The ultrasound images generated by the portable imaging device may be used to determine the location of the target artery to facilitate intervention (e.g., application of a tourniquet, providing vascular treatment, etc.) in-situ prior to arrival at the medical treatment facility, such as at the scene of the emergency medical event, during transport, and/or the like.

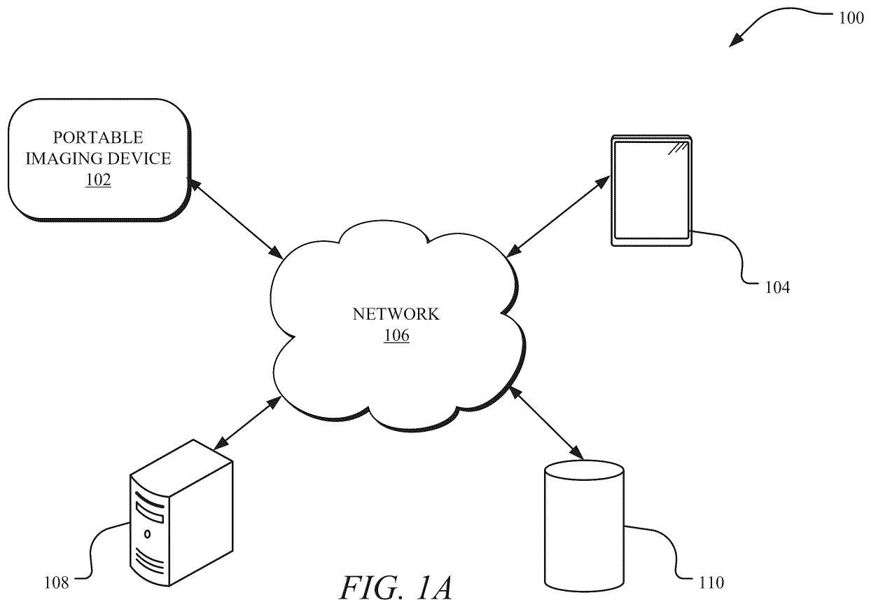

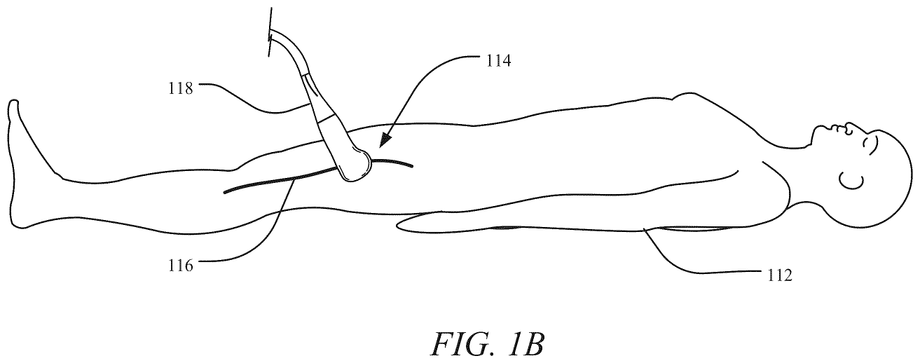

To begin a detailed description of an example system 100 for portable medical imaging, reference is made to FIGS. 1A-1B. In some implementations, the system 100 includes a portable imaging system 102 in communication with a user device 104. The portable imaging system 102 may be in communication with the user device 104 using a wired connection and/or wireless connection. For example, the portable imaging system 102 may be in communication with the user device 104 over a network 106.

The user device 104 may be any form of computing device capable of interacting with the portable imaging system 102, such as a personal computer, portable computer, workstation, mobile device, smartphone, tablet, wearable, heads-up display (HUD), and/or the like. In some examples, the portable imaging device is a small, portable, device configured to capture two-dimensional data, such as ultrasonic image data, to display one or more images (e.g., ultrasonic images) using the user device 104. In some examples, the user device 104 is integrated into the portable imaging system 102. The portable imaging device 102 and/or the user device 104 may be in communication with other computing devices, such as one or more data storage devices 108 and/or a server 110, to implement portable medical imaging of a patient 112 in response to an emergency medical event in various environments.

In some examples, the server 110 may host a website or an application that is executed or otherwise presented by the portable imaging device 102 and/or the user device 104. The website and/or application may provide access to a patient treatment system that facilitates communication with and operation of the portable imaging device 102. The patient treatment system may further capture patient data associated with an emergency medical event and communicates the patient data to a treatment system at a medical treatment facility to continue patient care upon arrival at the medical treatment facility. The patient treatment system may provide access to other computing components and/or other medical data via the network 106. The server 110 may be a single server, a plurality of servers with each such server being a physical server or a virtual machine, or a collection of both physical servers and virtual machines. In some examples, a cloud hosts one or more components of the patient treatment system. The user device 104, the server 110, and other resources connected to the network 106 may access one or more other servers to access to one or more websites, applications, web services interfaces, storage devices, computing devices, or the like that are used for patient treatment and/or operation of the portable imaging device 102. The server 110 may also host a search engine that the patient treatment system uses for accessing, searching for, and modifying patient data, medical intelligence, and/or other data.

The user device 104 may include a display, which may be in the form of a touch-sensitive display screen (“touchscreen”). Using the touchscreen, a medical provider, such as a member of an EMT team may control operation of the portable imaging device 102 and examine one or more images of the patient 112 captured using the portable imaging device 102.

In some implementations, the portable imaging device 102 may facilitate portable medical imaging in response to an emergency medical event, such as a critical injury, to evaluate the patient 112 for diagnosis and treatment. The portable imaging device 102 may be configured for deployment at various locations for imaging the patient 112 in-situ during the course of an emergency medical response. For example, the portable imaging device 102 may be used at locations remote from and/or within a medical treatment facility, such as a hospital. Locations remote from the medical treatment facility may include, for example, a scene of the emergency medical event and/or during transport from the scene to the medical treatment facility in an emergency vehicle. In this manner, the portable imaging device 102 facilitates imaging of the patient 112 where the patient 112 is located and as needed, and the portable imaging device may be used to diagnose and begin treatment in response to the emergency medical event before the patient 112 arrives at a medical treatment facility.

The portable imaging device 102 may be configured to provide two-dimensional imaging and/or three-dimensional imaging. In some examples, the portable imaging device 102 is configured to emit and detect ultrasonic waves, which may be visualized as one or more images on a display of the user device 104. The ultrasonic imaging may include, without limitation, B-mode, M-mode, color Doppler, spectral Doppler, and/or other outputs. The portable imaging device 102 may further be configured to obtain, without limitation, temperature, pressure, moisture, EKG signals, electrical signals, or other information indicative of patient condition. In some implementations, the portable imaging device 102 is a small, portable, ultrasound imaging device that can be used to locate a target artery 116 (e.g., a femoral artery) using image processing software that generates one or more images using ultrasonic data obtained during a scan of a target area 114 (e.g., a leg) of a body of the patient 112.

Ultrasonic imaging may be used to visualize blood flow, including blockages, sources of blood loss, and/or the like, as well as generally visualizing internal anatomical structures of the patient, such as vascular structures, cardiac structures, organs, etc. For example, the portable imaging device may include an image generation device 118, such as an ultrasonic probe, to capture ultrasound images of the target artery 116 within the target area 114. The target artery 116 may be a source of bleeding in some examples. Often, it is challenging to determine where a particular artery is located within a target area (e.g., where the femoral artery is located within a patient leg), letalone an artery that is a source of blood loss for treatment. The ultrasound images generated by the portable imaging device 102 may be used to determine the location of the target artery 116 to facilitate intervention in-situ prior to arrival at the medical treatment facility, such as at the scene of the emergency medical event, during transport, and/or the like. Such intervention may include, for example, application of a tourniquet and/or providing vascular treatment.

In some implementations, the image generation device 118 may be positioned on the target area 114 of the patient 112 and autonomously capture ultrasonic imaging data of the target area 114, including vascular structures, such as the target artery 116. For example, an imaging plane of the image generation device 118 may be autonomously adjusted to provide one or more views of the target area 114 in locating the target artery 116. The image generation device 118 and capture of ultrasonic image data may be further controlled using physical adjustors of the image generation device 118, the user device 104 (e.g., a touchscreen), and/or controls of the portable image system 102. For example, the image generation device 118 may be rotationally or translationally adjusted relative to a surface of the target area 114 of the patient 112. The image generation device 118 may capture ultrasonic imaging data from which one or more ultrasonic images are generated. The one or more ultrasonic images are rendered for display using the user device 104. By viewing the one or more ultrasonic images on the user device 104, a medical provider, such as an EMT, may locate the target artery 116 within the target area 114 of the patient 112.

The examples discussed herein with respect to FIGS. 1A-5 refer to portable imaging of the patient 112 using ultrasonic image data to locate the target artery 116 in the target portion 114 of the body of the patient 112 in an environment that is remote from a medical treatment facility, such as at the scene of an emergency medical event and/or during transport to the medical treatment facility. However, it will be appreciated by those skilled in the art that the presently disclosed technology is applicable to other imaging data, other anatomical structures, and other environments.

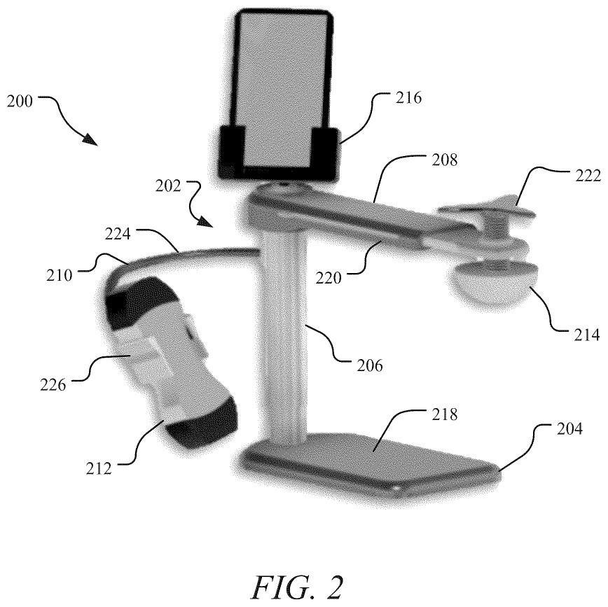

Referring to FIG. 2, an example system 200 for generating an image using a portable imaging device 202, is shown. The portable imaging device 202 is an example of the portable imaging device 102 described in FIG. 1 and configured to capture imaging data, such as ultrasonic imaging data, of the target area 114 of the patient 112. In an implementation, the system 200 is disposed relative to the target area 114 of the patient 112, such as, for example, an arm, leg, and/or other body part, to generate medical images, such as, for example, ultrasonic images of vascular structures, such as the target artery 116. Thus, the system 200 provides for portable vascular treatment by locating the target artery 116, such as, for example, a femoral artery. In an implementation, the portable imaging device 202 includes a base 204, a mounting post 206, an extension arm 208, an image generation device mount 210, an image generation device 212, a compression block 214, and a display mount 216.

In an implementation, the base 204 includes a base surface 218 to cooperatively define a treatment area with the extension arm 208. In an implementation, the base surface 218 is a substantially planar surface. In another implementation, the base surface 218 is curved to correspond with the body part of the patient 112. Although the base 204 is illustrated with a rectangular shape, the disclosure is not limited as such, and any suitable shape may be used.

In an implementation, the mounting post 206 is coupled to the base 204. In an implementation, the mounting post 206 extends from the base surface 218. In an implementation, the mounting post 206 extends in a substantially perpendicular direction to the base surface 218. In an implementation, the mounting post 206 is offset from a center of the base surface 218, such that the base 204 is cantilevered relative to the mounting post 206.

In an implementation, the extension arm 208 is coupled to the mounting post 206. In an implementation, the extension arm 208 extends substantially perpendicularly to the mounting post 206. In an implementation, the extension arm 208 is cantilevered relative to the mounting post 206. In an implementation, the extension arm 208 includes an extension arm surface 220 to cooperatively define a treatment area with the base surface 218. In this implementation, the extension arm surface 220 is substantially parallel to the base surface 218 In an implementation, the extension arm surface 220 is a substantially planar surface. In another implementation, the extension arm surface 220 is curved to correspond with the body part of the patient 112. Although the extension arm surface 220 is illustrated with a rectangular shape, the disclosure is not limited as such, and any suitable shape may be used.

In an implementation, the compression block 214 is coupled to the extension arm 208 via an actuator 222. In an implementation, the actuator 222 is a threaded knob, lever, etc. Actuation of the actuator 222, such as rotation of the threaded knob, for example, causes the compression block 214 to move relative to the extension arm 208 and the base 204. Thus, when a portion of the patient 112 is disposed within the treatment area, the compression block 214 can be moved by the actuator 222 to engage the portion of the patient 112 to restrict movement of the portable imaging device 202 relative to the patient 112 and to apply a compression force to the portion of the patient 112. In an implementation, the compression block 214 includes a ball, a disc, a block, etc. In an implementation, the compression block 214 is coated in a plastic or rubber material.

In an implementation, the image generation device mount 210 is coupled to at least one of the base 204, the mounting post 206, or the extension arm 208. In an implementation, the image generation device mount 210 includes a flexible arm 224 and a clamp 226. In an implementation, the flexible arm 224 is a gooseneck. The flexible arm 224 enables a medical provider to position and retain the image generation device 212 in any desired position, thereby allowing for both hands to be used during treatment procedures. In an implementation, the clamp 226 applies a force to the image generation device 212 to retain the image generation device 212 to the image generation device mount 210. In an implementation, the image generation device 212 is removable from the clamp 226 by overcoming the force.

In an implementation, the image generation device 212 generates image data. In an implementation, the image generation device 212 is an ultrasound probe that generates ultrasonic image data. In an implementation, the image generation device 212 is an autonomous ultrasound device. In an implementation, the image data allows for the location of one or more arteries, including the target artery 116, for a vascular treatment.

In an implementation, the display mount 216 is rotatably coupled to the base 204, the extension arm 208 or the mounting post 206. In an implementation, the display mount 216 can at least partially rotate relative to the extension arm 208 or the mounting post 206. The display mount 216 is operable to support a display, such as, for example, the user device 104. In an implementation, the user device 104 is communicatively coupled to the image generation device 212 via a wired or wireless connection, such that a display of the user device 104 presents a representation of image data generated by the image generation device 212. In an implementation, the user device 104 is a mobile device and/or tablet.

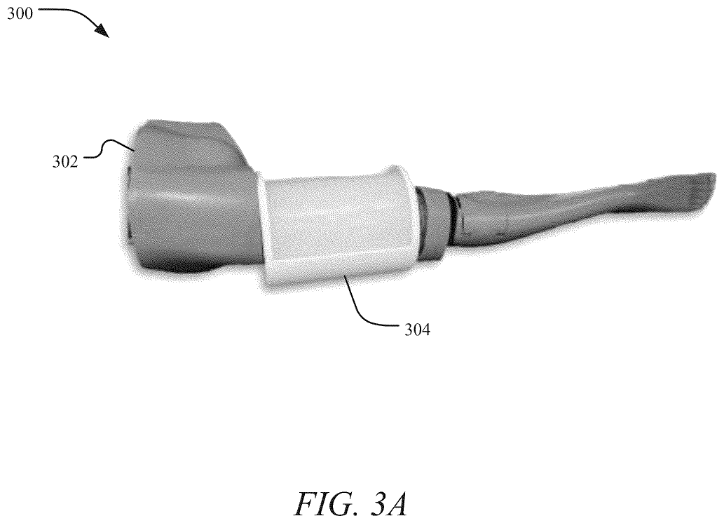

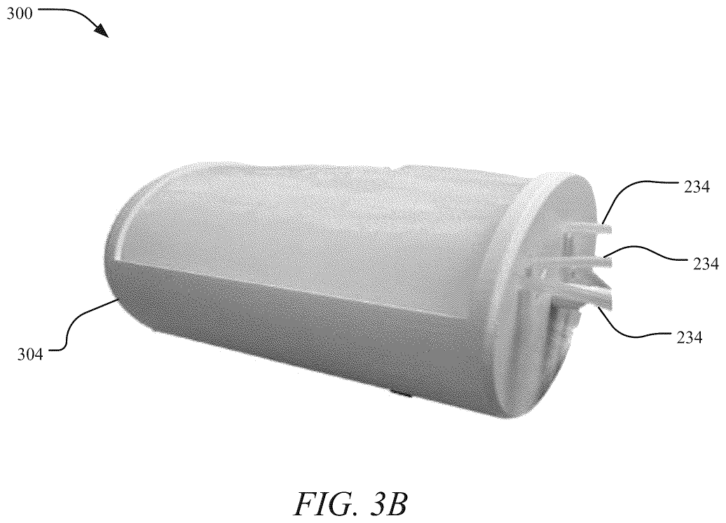

FIGS. 3A and 3B illustrate an example training system 300. In an implementation, the training system 300 simulates a target artery of a patient and can be used by an operator of the portable imaging device 202, such as a medical provider, to practice imaging. In an implementation, the training system 300 includes a body 302 and an artery simulator assembly 304.

In an implementation, the body 302 simulates a body part of a patient, such as, for example, an arm, leg, and/or other target areas. In an implementation, the body 302 includes a reservoir (not shown) that stores a blood simulating fluid for the artery simulator assembly 304.

In an implementation, the artery simulator assembly 304 is one of a plurality of artery simulator assemblies coupled to the body 302. Each of the plurality of pump assemblies correspond to different types of patients. In an implementation, the artery simulator assembly 304 includes a plurality of tubes 306, a pump (not shown), and a pump controller (not shown). The pump is controlled by the pump controller to cause fluid to flow from the reservoir via the plurality of tubes 306 to simulate blood flow. During training, the artery simulator assembly 304 is disposed in the treatment area of the portable imaging device 202 and is engaged by the compression block 214 to restrict movement therein. In an implementation, the pump is powered by a battery.

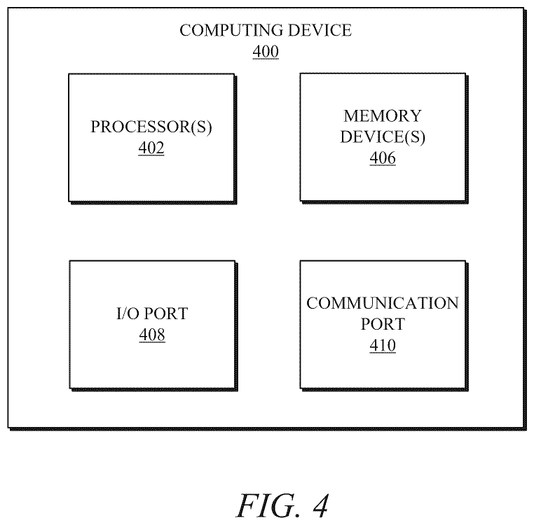

Turning to FIG. 4, a system 400 to process imaging data and generate output data can include one or more computing devices 402 for performing the techniques discussed herein. In one implementation, the one or more computing devices 402 are incorporated into the system 100 and/or the system 200, such as the portable imaging device 102, the user device 104, the portable imaging device 202, the image generation device 212, the server 110, and/or other computing devices to execute a software application and/or a module or algorithmic component of software to process the image data generated by the image generation device 212 to generate output data, such as an image, to be displayed on the display.

In some instances, the computing device 402 can include a computer, a personal computer, a desktop computer, a laptop computer, a terminal, a workstation, a server device, a cellular or mobile phone, a mobile device, a smart mobile device, a tablet, a wearable device (e.g., a smart watch, smart glasses, a smart epidermal device, etc.) a multimedia console, an Internet-of-Things (IoT) device, a smart home device, a medical device, a virtual reality (VR) or augmented reality (AR) device, and/or the like. It will be appreciated that specific implementations of these devices may be of differing possible specific computing architectures not all of which are specifically discussed herein but will be understood by those of ordinary skill in the art.

The computing device 402 may be a computing system capable of executing a computer program product to execute a computer process. Data and program files may be input to the computing device 402, which reads the files and executes the programs therein. Some of the elements of the computing device 402 include one or more processors 404, one or more memory devices 406, and/or one or more ports, such as input/output (IO) port(s) 408 and communication port(s) 410. Additionally, other elements that will be recognized by those skilled in the art may be included in the computing device 402 but are not explicitly depicted in FIG. 4 or discussed further herein. Various elements of the computing device 402 may communicate with one another by way of the communication port(s) 410 and/or one or more communication buses, point-to-point communication paths, or other communication means.

The processor 404 may include, for example, a central processing unit (CPU), a microprocessor, a microcontroller, a digital signal processor (DSP), and/or one or more internal levels of cache. There may be one or more processors 404, such that the processor 404 comprises a single central-processing unit, or a plurality of processing units capable of executing instructions and performing operations in parallel with each other, commonly referred to as a parallel processing environment.

The computing device 402 may be a conventional computer, a distributed computer, or any other type of computer, such as one or more external computers made available via a cloud computing architecture. The presently described technology is optionally implemented in software stored on the data storage device(s) such as the memory device(s) 406, and/or communicated via one or more of the I/O port(s) 408 and the communication port(s) 410, thereby transforming the computing device 402 in FIG. 4 to a special purpose machine for implementing the operations described herein. Moreover, the computing device 402, as implemented in the system 200, receives various types of input data (e.g., the image data) and transforms the data through various stages of the data flow into new types of data files (e.g., output data, display data, etc.). Moreover, these new data files are transformed further into output data and sent to the computing device 402 to generate a display indicating the image data, which enables the computing device 402 to do something it could not do before-generating ultrasonic images using a portable imaging device in-situ at locations remote from a medical treatment facility.

Additionally, the systems and operations disclosed herein represent an improvement in the technical field of medical imaging. For instance, the system 200 can generate ultrasonic images using a portable imaging device. Moreover, the image data can be leveraged to provide a highly effective vascular treatment. These techniques are rooted in technology and could not have existed prior to the advent of medical imaging.

The one or more memory device(s) 406 may include any non-volatile data storage device capable of storing data generated or employed within the computing device 402, such as computer executable instructions for performing a computer process, which may include instructions of both application programs and an operating system (OS) that manages the various components of the computing device 402. The memory device(s) 406 may include, without limitation, magnetic disk drives, optical disk drives, solid state drives (SSDs), flash drives, and the like. The memory device(s) 406 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory device(s) 406 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the memory device(s) 406 which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computing device 402 includes one or more ports, such as the I/O port(s) 408 and the communication port(s) 410, for communicating with other computing, network, or vehicle computing devices. It will be appreciated that the I/O port 408 and the communication port 410 may be combined or separate and that more or fewer ports may be included in the computing device 402.

The I/O port 408 may be connected to an I/O device, or other device, by which information is input to or output from the computing device 402. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing device 402 via the I/O port 408. Similarly, the output devices may convert electrical signals received from the computing device 402 via the I/O port 408 into signals that may be sensed as output by a human, such as sound, light, and/or touch. The input device may be an alphanumeric input device, including alphanumeric and other keys for communicating information and/or command selections to the processor 404 via the I/O port 408. The input device may be another type of user input device including, but not limited to direction and selection control devices, such as a mouse, a trackball, cursor direction keys, a joystick, and/or a wheel; one or more sensors, such as a camera, a microphone, a positional sensor, an orientation sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing device 402 via the I/O port 408. For example, an electrical signal generated within the computing device 402 may be converted to another type of signal, and/or vice-versa. In one implementation, the environment transducer devices sense characteristics or aspects of an environment local to or remote from the computing device 402, such as, light, sound, temperature, pressure, magnetic field, electric field, chemical properties, physical movement, orientation, acceleration, gravity, and/or the like.

In one implementation, the communication port 410 is connected to a network(s) so the computing device 402 can receive network data useful in executing the methods and systems set out herein as well as transmitting information and network configuration changes determined thereby. Stated differently, the communication port 410 connects the computing device 402 to one or more communication interface devices configured to transmit and/or receive information between the computing device 402 and other devices by way of one or more wired or wireless communication networks or connections. Examples of such networks or connections include, without limitation, Universal Serial Bus (USB), Ethernet, Wi-Fi, Bluetooth®, Near Field Communication (NFC), and so on. One or more such communication interface devices may be utilized via the communication port 410 to communicate with one or more other machines, either directly over a point-to-point communication path, over a wide area network (WAN) (e.g., the Internet), over a local area network (LAN), over a cellular network (e.g., third generation (3G), fourth generation (4G), Long-Term Evolution (LTE), fifth generation (5G), etc.) or over another communication means. Further, the communication port 410 may communicate with an antenna or other link for electromagnetic signal transmission and/or reception.

In an example, the software, modules, services, and operations discussed herein may be embodied by instructions stored on the memory device(s) 406 and executed by the processor 404.

The system set forth in FIG. 4 is but one possible example of a computing device 402 or computer system that may be configured in accordance with aspects of the present disclosure. It will be appreciated that other non-transitory tangible computer-readable storage media storing computer-executable instructions for implementing the presently disclosed technology on a computing system may be utilized. In the present disclosure, the methods disclosed may be implemented as sets of instructions or software readable by the computing device 402.

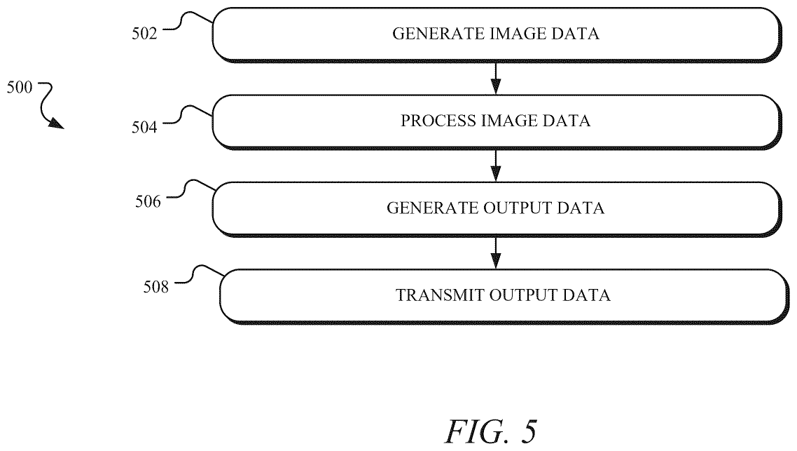

FIG. 5 depicts an example method 500 for generating an image using a portable imaging device, such as the portable imaging device 102 and/or the portable imaging device 202. The portable imaging device may be in communication with a user device via a wired or wireless connection.

At operation 502, the method 500 can generate image data using the image generation device 212 of the portable imaging device 202 that is positioned relative to a portion of a patient, such as, for example, an arm, leg, etc. of the patient.

At operation 504, the method 500 can process the image data using an image processing algorithm, such as, for example, an ultrasonic image processing algorithm.

At operation 506, the method 500 can generate output data, such as, for example display data.

At operation 508, the method 500 can transmit the output data to a computing device, to cause a display of the computing device to indicate the output data as an image, such as, for example, an ultrasonic image. In an implementation, operations 502-508 may repeat, thereby continuously updating the displayed image to present a real-time or near real-time image.

It is to be understood that the specific order or hierarchy of operations in the methods depicted in FIG. 5 and throughout this disclosure are instances of example approaches and can be rearranged while remaining within the disclosed subject matter. For instance, any of the operations depicted in FIG. 5 may be omitted, repeated, performed in parallel, performed in a different order, and/or combined with any other of the operations depicted in FIG. 5 or discussed herein.

Furthermore, any term of degree such as, but not limited to, “substantially,” as used in the description and the appended claims, should be understood to include an exact, or a similar, but not exact configuration. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A portable imaging system comprising:
a portable medical imaging device including:
a base including a base surface;
a mounting post coupled to base and extending from the base surface;
an extension arm coupled to the mounting post and including an extension arm surface that cooperatively forms a treatment area with the base surface; and
an image generation device mount coupled to at least one of the base, the mounting post, or the extension arm.

2. The system of claim 1, further comprising:
an image generation device coupled to the image generation device mount, the image generation device configured to generate image data.

3. The system of claim 2, wherein the image data is ultrasonic image data.

4. The system of claim 2, wherein the portable medical imaging device further includes:
a display mount rotatably coupled to at least one of the base, the mounting post, or the extension arm.

5. The system of claim 4, wherein the portable medical imaging device is in communication with a user device having a display disposed in the display mount, the display configured to present a representation of the image data generated by the image generation device.

6. The system of claim 1, wherein the portable medical imaging device further includes:
a compression block coupled to the extension arm via an actuator, wherein actuation of the actuator causes the compression block to move relative to the extension arm and the base.

7. The system of claim 6, wherein the actuator is a threaded knob.

8. The system of claim 1, wherein the image generation device mount includes a flexible arm and a clamp.

9. The system of claim 8, wherein the flexible arm is a gooseneck.

10. The system of claim 1, wherein the base surface and the extension arm surface are each a planar surface.

11. The system of claim 1, further comprising:
a training system including:
a body; and
an artery simulator assembly.

12. The system of claim 11, wherein the artery simulator assembly includes a pump and a plurality of tubes.

13. The system of claim 11, wherein the artery simulator assembly is one of a plurality of artery simulator assemblies.

14. A portable medical imaging device comprising:
a base;
a mounting post coupled to and extending from the base;
an extension arm coupled to the mounting post cooperatively forming a treatment area with the base; and
an image generation device mount coupled to at least one of the base, the mounting post, or the extension arm, the image generation device mount configured to couple to an image generation device, the image generation device configured to generate image data.

15. The portable medical imaging device of claim 14, further comprising:
a display mount coupled to at least one of the base, the mounting post, or the extension arm, the display mount configured to receive a display, the display communicatively coupled to the image generation device and configured to present a representation of the image data generated by the image generation device.

16. The portable medical imaging device of claim 14, further comprising:
a compression block coupled to the extension arm and including an actuator, wherein actuation of the actuator causes the compression block to move relative to the extension arm and the base.

17. The portable medical imaging device of claim 16, wherein the actuator is a threaded knob.

18. The portable medical imaging device of claim 14, wherein the image generation device mount includes a flexible arm and a clamp.

19. The portable medical imaging device of claim 18, wherein the flexible arm is a gooseneck.

20. One or more tangible non-transitory computer-readable storage media storing computer-executable instructions for performing a computer process on a computing system, the computer process comprising:
receiving image data captured using an image generation device, the image generation device coupled to a portable medical imaging device via an image generation device mount, the image generation device mount coupled to at least one of a base, a mounting post, or an extension arm of the portable medical imaging device;
processing the image data to generate processed image data;
generating output data using the processed image data; and
transmitting the output data to a display to cause an output of a representation of the output data.
